(19) United States
(12) Patent Application Publication
(10) Pub. No.: US 2026/0165937 A1
(43) Pub. Date: Jun. 18, 2026

(54) PEPTIDES FOR KERATIN REPAIR AND COMPOSITIONS THEREOF

(71) Applicant: Bio Genetic Technology LLC, North Bergen, NJ (US)

(72) Inventor: Yonghua Tang, North Bergen, NJ (US)

(73) Assignee: Bio Genetic Technology LLC, North Bergen, NJ (US)

(21) Appl. No.: 18/982,859

(22) Filed: Dec. 16, 2024

(51) Int. Cl. A61K 8/64; A61Q 5/00; C07K 7/06

(52) U.S. Cl. CPC A61K 8/64; A61Q 5/00; C07K 7/06

(57) ABSTRACT

Peptides for keratin repair and compositions incorporating such peptides are disclosed herein. The peptides and compositions incorporate specific amino acid sequences that contain both natural canonical amino acids and synthetic amino acid groups that mimic portions of α and β-keratin molecules present in human hair, skin, and nails. The disclosed peptides are engineered to self-combine with the naturally occurring α and β-keratin molecular complexes found in hair, skin, and nails, wherein the hair, skin, or nail structures are strengthened through application of compositions containing the keratin-repair peptides. The amino acid sequences of the peptides disclosed herein complex with keratin in hair to increase hair breaking strength greater than prior art protein hydrolysates used in existing hair care products.

### TECHNICAL FIELD

The disclosures herein relate to compositions for skin and hair care. Specifically, the disclosed invention relates to synthetic non-naturally occurring peptides to facilitate keratin repair, related compositions, and methods of use thereof.

### INCORPORATION BY REFERENCE OF SEQUENCE LISTING

Accompanying this filing is a Sequence Listing entitled “KProSequence_ST26.xml,” created on 16 Dec. 2024 and having 100 kilobytes of data. The sequence listing is hereby incorporated herein by reference in its entirety for all purposes.

### BACKGROUND

Keratin is a mammalian structural protein found in hair and skin, and nails. With considerable variation between types and subtypes, keratin comprises a chain of amino acids between about 350 and about 620 long. There are about twenty (20) subtypes grouped into alpha (α), beta (β), and gamma (γ) types. Different relative compositions of α, β, and γ keratin give different anatomic structures their constituent properties, such as soft, flexible hair and skin, or hard, somewhat rigid nails, claws, and hooves, for example.

Alpha keratin (“soft keratin”) is the primary keratin constituent of hair and is formed from a base unit of two right-handed helical peptide strands combined into a left-handed helical dimer. The amino acid composition of alpha keratin contains a high proportion of cystine available for disulfide bonds between adjacent peptides substantially contributing to stability of the dimers. The paired-strand helical dimers, in turn, self-polymerize, aligning end-to-end with other dimers through hydrogen bonding between N and C-terminal groups forming elongate fibrils or “protofilaments.” Overlapping protofilaments align and are bound by additional disulfide and hydrogen bonds creating many filaments grouped into overlapping bundles, and so on. Healthy hair is resilient, soft, and can be axially stretched without breaking, largely thought to be a consequence of the foundational helical structure and overlapping interactions between the parallel alpha keratin filament groups comprising its structure. Alpha keratin is also the primary keratin forming the epidermis of human skin.

Beta keratin (“hard keratin”) is the primary keratin component of nails, claws and hooves, feathers, and the epidermis of birds and reptiles. Human hair also comprises beta keratin, albeit in much smaller amounts. The structure of beta keratin consists of peptide chains folded back-and-forth upon themselves in a pleated, co-planar manner to form a sheet-like structure (beta sheet). Strong hydrogen bonds between C═O and N—H groups of adjoining lengths of the amino acid chains create and stabilize the beta-sheet secondary structure. Overlapping “stacked” sheets form scale-like protein layers and add rigidity to structures.

Human hair is considered an epithelial tissue. Hair contains no nerves or blood vessels and is not an organ. Hair functions to protect underlying skin and helps the body regulate temperature by retaining heat and enhancing evaporative heat loss by dispersing sweat. The average human scalp has around 1×10⁵ hairs. Hair can be less than a centimeter in length, or well over 100 centimeters long in some individuals.

Human hair has three histologic divisions: the cuticle, cortex, and medulla. The cortex forms the majority the hair's bulk and is largely comprised of fibrillar bundles of alpha keratin in the cortex. These cortical fibrillar keratin bundles are critical to the structure, appearance, and resilience of hair. The cortex surrounds the thin-core medulla containing disorganized alpha keratin fibrils and a large percentage of globular-shaped γ keratin. Formed largely from beta keratin, the cuticle is the outermost layer and has the appearance of overlapping scales encasing the cortex. The cuticle functions to protect the fibril bundles of the cortex from physical and chemical damage. Beta keratin forming the cuticle has a high proportion of hydrophobic aliphatic amino acid residues and resists water loss and infiltration of other materials into or from the hair fiber.

As noted, disulfide and hydrogen bonds are necessary for these secondary and tertiary keratin protein structures. Perming and dying processes disrupt these non-covalent chemical bonds, changing the hair's shape, color, elasticity, resilience, and strength. A healthy keratin structure gives hair good elasticity, allowing it to stretch when pulled and return to its original state upon release. When damaged, the elasticity of the hair is reduced, making it more likely to break under the same amount of stress. Keratin damage also leads to the loss of hair's natural oil protection layer, making the hair appear dull, dry, and brittle. This type of damage may result from the use of heat-styling devices such as blow dryers and straighteners, or chemical treatments such as dying and perming. Once the hair cuticle is compromised, exposed fibrillar bundles can fray or split, and hair breakage may occur from simple hygienic activities such as washing, combing, and brushing. Additionally, the β-keratin cuticular scales are reflective, giving health hair a glossy appearance. Damage to the cuticle creates surface irregularities making hair appear dull and rough. The uneven surface of damages hair also makes hair more difficult to brush, increases breakage with brushing or combing, and increases tangling.

Existing hair and skin care products may contain hydrolyzed keratin, usually β-keratin from cow hooves and horns, in a base composition containing emollients and hydrators. Hydrolyzed keratin, however, does not necessarily incorporate into the specific α and β-keratin molecules present in human hair and skin, presumably because of non-homologous amino acid sequences, incorrect proportion of cystine residues, and other differences.

Because of these and other problems, there is a need for hair and skin care products incorporating peptide molecules for treatment of damaged keratin to improve the function and appearance of human hair.

### BRIEF SUMMARY

Disclosed herein is a non-naturally occurring peptide characterized by the amino acid sequence H-Gly-Gly-Cys-[X_{(N<5)}-Aib_{(M>1)}]₆-Cys-OH where X is a non-polar amino acid and Aib is 2-aminoisobutyric acid. In some embodiments, the nonpolar amino acid is taken from the group consisting of glycine, alanine, valine, leucine, and isoleucine. The group [X_{(N<5)}-Aib_{(M>1)}]₆ is any combination of six total amino acid residues, with no greater than five (5) non-polar amino acids and at least one (1) 2-aminoisobutyric acid residue.

In some embodiments, the peptide amino acid sequence is H-Gly-Gly-Cys-Ala-Val-Gly-Leu-Ala-Aib-Cys-OH (SEQ_ID 2). In some embodiments, the peptide amino acid sequence is H-Gly-Gly-Cys-Aib-Val-Aib-Leu-Ala-Aib-Cys-OH (SEQ_ID 3). In some embodiments, the peptide amino acid sequence is H-Gly-Gly-Cys-Aib-Aib-Aib-Aib-Aib-Aib-Cys-OH (SEQ_ID) 4.

In some embodiments, a single polar amino acid replaces a single non-polar amino acid. In some embodiments, the single polar amino acid is threonine and the single non-polar amino acid is the amino acid in position 3.

Disclosed is a peptide having the amino acid sequence H-Gly-Gly-Cys-Ala-Val-Gly-Leu-Ala-Gly-Cys-OH (SEQ_ID5).

In some embodiments, the peptide is in combination with an excipient from the list of excipients consisting of cetearyl alcohol, lauric acid, ammonium lauryl sulfate, aloe vera extract, cocoamide, cocamidopropyl betaine, cocoa butter, coconut oil, glycerin, jasmine oil or extract, jojoba oil or extract, sodium lauryl sulfate, eucalyptus oil or extract, and tea tree oil or extract and forms a hair care product. In some embodiments, the peptide is in combination with an excipient from the list of excipients consisting of aloe vera, beta carotene, cocoa butter, coconut oil, cucumber extract, hyaluronic acid, retinoic acid, tretinoin, retinol, shea butter, soy lecithin, and vitamin E including any of alpha/beta/gamma/delta tocopherol and/or alpha/beta/gamma/delta tocotrienol and forms a skin care or a nail care product.

The foregoing and other features and advantages of the invention will be apparent to those of ordinary skill in the art from the following more particular description of the invention and the accompanying drawings.

### BRIEF DESCRIPTION OF THE DRAWINGS

FIG. 1 is a bar graph representation of results of a hair tensile strength test between hair treated with a prior art peptide composition, a composition containing a peptide for keratin repair, and a non-peptide containing control solution; and

FIG. 2 is a bar graph representation of results of a test comparing the effects of various peptide compositions on hair tensile strength with a non-peptide containing control solution.

### DETAILED DESCRIPTION

Various example embodiments of peptides for keratin repair are provided herein. The disclosed peptides incorporate non-polar amino acid residues, modified amino acids, and cystine in various combinations. Aqueous solutions of these peptides exhibit excellent properties with respect to repairing damaged hair by increasing tensile breaking force.

### Definitions

As used herein, “2-aminoisobutyric acid” and “Aib” mean the amino acid compound having the structural formula H₂N—C(CH₃)₂—COOH and PubChem CIC #6119.

As used herein, “amino acid residue” means a single amino acid molecule incorporated into a peptide.

As used herein, “non-polar amino acid” means any stereoisomer of any of alanine, glycine, isoleucine, leucine, methionine, phenylalanine, proline, or valine. “Hydrophobic amino acid” means a non-polar amino acid or 2-aminoisobutyric acid.

As used herein, “modified amino acid,” non-canonical amino acid, or “atypical amino acid,” any of which terms may be used interchangeably, mean an organic molecule containing both an amine and a carboxylic acid group that are not directly encoded by the native “wild-type” human genome.

The present disclosure encompasses compositions and methods for treading damaged keratin structures, such as hair, skin, and nails, for example. In some embodiments, the compositions include a non-naturally occurring peptide characterized by SEQ ID NO: 1, namely, the amino acid sequence (H-Gly-Gly-Cys-[X(N<5)-Aib(M>1)]6-Cys-OH where X is a non-polar amino acid and Aib is 2-aminoisobutyric acid. Compositions incorporating the peptide having SEQ ID NO: 1 are configured to repair hair, skin, nails, and other keratin-based structures, in some embodiments. In some embodiments, the nonpolar amino acid is taken from the group consisting of glycine, alanine, valine, leucine, and isoleucine. The group [X(N<5)-Aib(M>1)]6 is any combination of six total amino acid residues, with no greater than five (5) non-polar amino acids and at least one (1) 2-aminoisobutyric acid residue.

In some embodiments, the composition comprises a peptide having an amino acid sequence consisting of one of the group of amino acid sequences consisting of SEQ ID NO: 1, SEQ ID NO: 2, SEQ ID NO: 3, and SEQ ID NO: 4.

In some embodiments, the composition comprises a peptide having an amino acid sequence consisting of one of the group of amino acid sequences consisting of SEQ ID NO: 5, SEQ ID NO; and 6, SEQ ID NO: 7.

In some embodiments, the composition comprises a peptide having a base sequence of H-Gly-Gly-Cys[X_{(N<5)} Aib_{(M>0)}]⁶-Cys-OH where N=3, M=3 and “X” N refers to the number of a non-polar amino acid from the group of non-polar amino acids consisting of glycine (Gly), alanine (Ala), valine (Val), leucine (Leu), isoleucine (Ile), proline (Pro), phenylalanine (Phe), tryptophan (Trp), methionine (Met), and 2-amino isobutyric acid (AiB); “N” refers to the number of non-polar amino acids “X”, and “M” refers to the number of Aib amino acid residues. In some embodiments, N=3 and M=3. In some embodiments M is less than 6, or less than 5, or less than 4. In some embodiments, M is greater than 4.

In some embodiments, the composition comprises a peptide having an amino acid sequence consisting of one of the group of amino acid sequences consisting of SEQ ID NOs: 8-108. In some embodiments, a total peptide concentration of the composition is the concentration of peptides defined by the SEQ ID NOs. disclosed herein.

In some embodiments, the keratin repair composition comprises a total peptide concentration by weight of between about 0.001 percent and about 0.05 percent. In some embodiments, the keratin repair composition comprises a total peptide concentration by weight of between about 0.001 percent and about 0.0025 percent. In some embodiments, the keratin repair composition comprises a total peptide concentration by weight of between about 0.0026 percent and about 0.050 percent. In some embodiments, the keratin repair composition comprises a total peptide concentration by weight of between about 0.0051 percent and about 0.075 percent. In some embodiments, the keratin repair composition comprises a total peptide concentration by weight of between about 0.0076 percent and about 0.099 percent.

In some embodiments, the keratin repair composition comprises a total peptide concentration by weight of between about 0.010 percent and about 0.015 percent. In some embodiments, the keratin repair composition comprises a total peptide concentration by weight of between about 0.015 percent and about 0.020 percent. In some embodiments, the keratin repair composition comprises a total peptide concentration by weight of between about 0.021 percent and about 0.030 percent. In some embodiments, the keratin repair composition comprises a total peptide concentration by weight of between about 0.031 percent and about 0.040 percent. In some embodiments, the keratin repair composition comprises a total peptide concentration by weight of between about 0.040 percent and about 0.050 percent.

In some embodiments, the keratin repair composition comprises a total peptide concentration by weight of between about 0.051 percent and about 0.075 percent. In some embodiments, the keratin repair composition comprises a total peptide concentration by weight of between about 0.076 percent and about 0.099 percent.

In some embodiments, the keratin repair composition comprises a total peptide concentration by weight of about 0.10 percent.

In some embodiments, the keratin repair composition comprises a total peptide concentration by weight of between about 0.11 percent and about 0.15 percent. In some embodiments, the keratin repair composition comprises a total peptide concentration by weight of between about 0.16 percent and about 0.25 percent. In some embodiments, the keratin repair composition comprises a total peptide concentration by weight of between about 0.26 percent and about 0.50 percent. In some embodiments, the keratin repair composition comprises a total peptide concentration by weight of between about 0.51 percent and about 0.75 percent. In some embodiments, the keratin repair composition comprises a total peptide concentration by weight of between about 0.76 percent and about 0.99 percent.

In some embodiments, the keratin repair composition comprises a total peptide concentration by weight of about 1.0 percent. In some embodiments, the keratin repair composition comprises a total peptide concentration by weight of greater than about 1.0 percent.

In some embodiments, the keratin repair composition is configured as a hair care product. In some embodiments the hair care product is formed by combining the keratin repair peptide with one or more excipients used in hair care products from the group of excipients consisting of cetearyl alcohol, lauric acid, ammonium lauryl sulfate, aloe vera extract, cocoamide, cocamidopropyl betaine, cocoa butter, coconut oil, glycerin, jasmine oil or extract, jojoba oil or extract, sodium lauryl sulfate, eucalyptus oil or extract, and tea tree oil or extract, for example. The keratin repair peptide and the one or more excipients are suspended and/or dissolved in a hydrophilic medium such as deionized water, in some embodiments. In some embodiments, the hair care product is formed as a gel, a cream, or an oil.

In some embodiments, the keratin repair composition is configured as a skin care product by combining the keratin repair peptide with one or more excipients use in skin car products from the group of excipients consisting of aloe vera, beta carotene, cocoa butter, coconut oil, cucumber extract, hyaluronic acid, retinoic acid, tretinoin, retinol, shea butter, soy lecithin, and vitamin E including any of alpha/beta/gamma/delta tocopherol and/or alpha/beta/gamma/delta tocotrienol. In some embodiments, the skin care product is formed as a gel, a cream, or an oil.

In some embodiments, the keratin repair composition is configured as a nail care product by combining the keratin repair peptide with one or more excipients use in nail care products from the group of excipients consisting of aloe vera, beta carotene, cocoa butter, coconut oil, glycerin, hyaluronic acid, soy lecithin, and vitamin E including any of alpha/beta/gamma/delta tocopherol and/or alpha/beta/gamma/delta tocotrienol. In some embodiments, the nail care product is formed as a gel, a cream, or an oil.

### Example 1: Comparison of Prior-Art Hair Care Peptide with Disclosed Composition

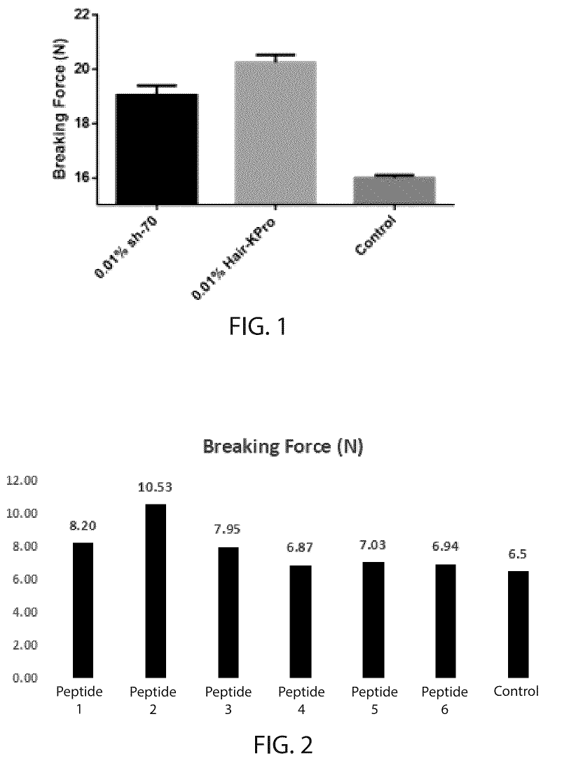

Tensile strength testing was performed to determine breaking force of hair fibers before and after chemical-induced damage. A digital electronic tester (50N Single Arm Microcomputer Control Electronic Testing Machine—Non-Metallic Material Tensile Machine sold by Kubrishan Lugong Precision Instrument Co., LTD; URL: https://www.hbzhan.com/st663026/product_24758811.html) was used to record breaking strength using the following method:
1. Standardized severely damaged hair strands were created by taking freshly cut human hair and treating with aqueous solutions of ammonia and hydrogen peroxide of various concentrations and durations.
2. The damaged strands were collected into clusters, each cluster containing twenty-five (25) individual damaged hair strands.
3. Three (3) test solutions denoted “A,” “B,” and “C” were prepared. Solution A consisted of a hydrolized protein hair care product from the prior art (0.01% SH-RD Protein Hair Mask sold by Shaanhonq International Cosmetics Corporation, Kaohshung Taiwan; URL: https://shrd.us/products/sh-rd-protein-hair-mask-2-37oz-70ml?srsltid=AfmBOorncS5soiPwxRWuaJRg-bEOKrORz3bhmeqGTmZ8JQGvAzmeanIY.); Solution B consisted of 0.01% solution of Gly-Gly-Cys-Ala-Val-Gly-Leu-Ala-Aib-Cys in deionized water; and Solution C consisted of deionized water containing no peptide.
4. Hair clusters with standardized damage (per step 1 above) were immersed in solutions A, B, and C for 60 minutes.
5. The clusters were removed from the Solutions and allowed to dry.
6. Each cluster was then loaded into the tension tester and subjected to gradually increasing tension until complete breakage of the cluster resulted and the tension value was recorded.
7. The test was performed for three times each for clusters treated with each treatment Solution used and the average breakage tension was recorded for clusters treated with each treatment Solution. The results are summarized in FIG. 1.

As shown in FIG. 1, both treatment solutions (A and B) containing peptides or protein hydrolysate substantially increased tensile breaking strength to the hair clusters versus treatment with deionized water containing no peptides. The average breaking force for the Solution A-treated hair was greater than the average breaking force for the hair treated with Solution B. Therefore, hair treated with [peptide/mixture] conferred significantly greater tensile strength than a simple protein hydrolysate.

### Example 2—Comparison of Six(6) Novel Peptide Compositions

A peptide “1” having a base sequence of H-Gly-Gly-Cys[X_{(N<5)} Aib_{(M>0)}]⁶-Cys-OH where “N” refers to the number of a non-polar amino acids from the group of non-polar amino acids consisting of glycine (Gly), alanine (Ala), valine (Val), leucine (Leu), isoleucine (Ile), proline (Pro), phenylalanine (Phe), tryptophan (Trp), and methionine (Met); and “M”, refers to the number of Aib amino acid residues was used to design the following peptide: H-Gly-Gly-Cys-Ala-Val-Gly-Leu-Ala-Aib-Cys-OH. (SEQ_ID2). The peptide (SEQ_ID2) was proportionally added to deionized water until a final concentration of 0.01% by weight was reached. The preparation was stirred well and set aside for use.

A peptide “2” having a base sequence of H-Gly-Gly-Cys[X_{(N<5)} Aib_{(M>0)}]⁶-Cys-OH where “N” refers to the number of a non-polar amino acids from the group of non-polar amino acids consisting of glycine (Gly), alanine (Ala), valine (Val), leucine (Leu), isoleucine (Ile), proline (Pro), phenylalanine (Phe), tryptophan (Trp), and methionine (Met); and “M”, refers to the number of Aib amino acid residues was used to design the following peptide: H-Gly-Gly-Cys-Aib-Val-Aib-Leu-Ala-Aib-Cys-OH. (SEQ_ID3). The peptide (SEQ_ID3) was proportionally added to deionized water until a final concentration of 0.01% by weight was reached. The preparation was stirred well and set aside for use.

A peptide “3” having a base sequence of (H)-Gly-Gly-Cys[X_{(N<5)} Aib _{(M>0)}]⁶-Cys-(OH) where “N” refers to the number of a non-polar amino acids from the group of non-polar amino acids consisting of glycine (Gly), alanine (Ala), valine (Val), leucine (Leu), isoleucine (Ile), proline (Pro), phenylalanine (Phe), tryptophan (Trp), and methionine (Met); and “M”, refers to the number of Aib amino acid residues was used to design the following peptide: H-Gly-Gly-Cys-Aib-Aib-Aib-Aib-Aib-Aib-Cys-OH-Leu-Ala-Aib-Cys-OH. (SEQ_ID4). The peptide (SEQ_ID4) was proportionally added to deionized water until a final concentration of 0.01% by weight was reached. The preparation was stirred well and set aside for use.

A peptide “4” consisting of the amino acid sequence: H-Gly-Gly-Cys-Ala-Val-Gly-Leu-Ala-Gly-Cys-OH (SEQ_ID5) was proportionally added to deionized water until a final concentration of 0.01% by weight was reached. The preparation was stirred well and set aside for use.

A peptide “5” consisting of the amino acid sequence: H-Gly-Gly-Cys-Ala-Val-Gly-Leu-Ala-Gly-Gly-Aib-Cys-OH (SEQ_ID6) was proportionally added to deionized water until a final concentration of 0.01% by weight was reached. The preparation was stirred well and set aside for use.

In some embodiments, a single polar amino acid residue replaces a single non-polar amino acid residue. In this example, a peptide “6” consisting of the amino acid sequence: H-Gly-Gly-Cys-Thr-Val-Gly-Leu-Ala-Aib-Cys-OH (SEQ_ID7) was proportionally added to deionized water until a final concentration of 0.01% by weight was reached. The preparation was stirred well and set aside for use.

The tensile break strength test utilizing the same method steps of Example 1 discussed above was performed on each of the six (6) sample compositions listed above and a control sample hair bundle treated with plain deionized water. The results are summarized in FIG. 2. The breaking strength of the treated hair bundles are shown in Table 1 below:

TABLE 1
BREAKING FORCE (N)

| | |
|---|---|
| Peptide 1 | 8.20 |
| Peptide 2 | 10.53 |
| Peptide 3 | 7.95 |
| Peptide 4 | 6.87 |
| Peptide 5 | 7.03 |
| Peptide 6 | 6.94 |
| Control | 6.50 |

As shown by FIG. 2 and the results listed above in Table 1, the tensile breaking strength of the samples from the examples of peptides used in a keratin repair composition, all compositions demonstrated a capacity for imparting increased tensile strength to the hair bundles, presumably through keratin repair. The chemically damaged hair treated with samples from all groups showed an increase in tensile strength. The increase from use of peptides 1, 2, and 3 were higher when compared to the other examples. This suggests that peptides formed using the base sequence of H-Gly-Gly-Cys[X_{(N<5)} Aib_{(M>0)}]⁶-Cys-OH where “N” refers to the number of a non-polar amino acids from the group of non-polar amino acids consisting of glycine (Gly), alanine (Ala), valine (Val), leucine (Leu), isoleucine (Ile), proline (Pro), phenylalanine (Phe), tryptophan (Trp), and methionine (Met); and “M”, refers to the number of Aib amino acid residues are superior to other peptides for incorporation into keratin repair compositions for use on hair.

Peptides for keratin repair and compositions incorporating such peptides have been disclosed herein. The peptides and compositions incorporate specific amino acid sequences that contain both natural canonical amino acids and synthetic amino acid groups that mimic portions of α and β-keratin molecules present in human hair and skin. The disclosed peptides self-combine with the naturally occurring α and β-keratin molecular complexes found in hair, skin, and nails, wherein the hair, skin, or nail structures are strengthened through application of compositions containing the keratin-repair peptides. The amino acid sequences of the peptides disclosed herein are engineered to complex with keratin in hair to increase hair breaking strength greater than prior art protein hydrolysates used in existing hair care products.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings herein above.

What is claimed is:

1. A peptide comprising the amino acid sequence: H-Gly-Gly-Cys-[X_{(N≤5)} Aib_{(M≥1}]⁶-Cys-OH wherein “X” is a non-polar amino acid from the group of non-polar amino acids consisting of Glycine (Gly), Alanine (5 Ala), Valine (Val), Leucine (Leu), and Isoleucine (Ile) and “[X_{(N≤5)} Aib_{(M≥1)}]⁶” denotes any combination of six amino acids comprising no more than five non-polar amino acids and at least one 2-aminoisobutyric acid (SEQ_ID 1).

2. The peptide of claim 1, wherein the amino acid sequence is H-Gly-Gly-Cys-Ala-Val-Gly-Leu-Ala-Aib-Cys-OH (SEQ_ID 2).

3. The peptide of claim 1, wherein the amino acid sequence is H-Gly-Gly-Cys-Aib-Val-Aib-Leu-Ala-Aib-Cys-OH (SEQ_ID 3).

4. The peptide of claim 1, wherein the amino acid sequence is H-Gly-Gly-Cys-Aib-Aib-Aib-Aib-Aib-Aib-Cys-OH (SEQ_ID) 4.

5. The peptide of claim 1, wherein a single polar amino acid replaces a single non-polar amino acid.

6. The peptide of claim 5, wherein the single polar amino acid is threonine and the single non-polar amino acid is the amino acid in position 3.

7. A peptide consisting of the amino acid sequence: H-Gly-Gly-Cys-Ala-Val-Gly-Leu-Ala-Gly-Cys-OH (SEQ_ID5).

8. The peptide of claim 1 in combination with an excipient from the list of excipients consisting of cetearyl alcohol, lauric acid, ammonium lauryl sulfate, aloe vera extract, cocoamide, cocamidopropyl betaine, cocoa butter, coconut oil, glycerin, jasmine oil or extract, jojoba oil or extract, sodium lauryl sulfate, eucalyptus oil or extract, and tea tree oil or extract.

9. The peptide of claim 1 in combination with an excipient from the list of excipients consisting of aloe vera, beta carotene, cocoa butter, coconut oil, cucumber extract, hyaluronic acid, retinoic acid, tretinoin, retinol, shea butter, soy lecithin, and vitamin E including any of alpha/beta/gamma/delta tocopherol and/or alpha/beta/gamma/delta tocotrienol.


SEQUENCE LISTING (111 sequences)

SEQ ID NO: 1 - protein, 5 aa, synthetic construct
  VAR_SEQ 4: note = [X(N<5)Aib(M>1)]6 where X=G, A, V, L, I
GGCXC

SEQ ID NO: 2 - protein, 10 aa, synthetic construct
  MOD_RES 9: note = Aib
GGCAVGLAXC

SEQ ID NO: 3 - protein, 10 aa, synthetic construct
  MOD_RES 4: note = Aib
  MOD_RES 6: note = Aib
  MOD_RES 9: note = Aib
GGCXVXLAXC

SEQ ID NO: 4 - protein, 10 aa, synthetic construct
  MOD_RES 4: note = Aib
  MOD_RES 5: note = Aib
  MOD_RES 6: note = Aib
  MOD_RES 7: note = Aib
  MOD_RES 8: note = Aib
  MOD_RES 9: note = Aib
GGCXXXXXXC

SEQ ID NO: 5 - protein, 10 aa, synthetic construct
GGCAVGLAGC

SEQ ID NO: 6 - protein, 12 aa, synthetic construct
  MOD_RES 11: note = Aib
GGCAVGLAGGXC

SEQ ID NO: 7 - protein, 10 aa, synthetic construct
  MOD_RES 9: note = Aib
GGCTVGLAXC

SEQ ID NO: 8 - protein, 13 aa, synthetic construct
GGCGAIBGAVLIC

SEQ ID NO: 9 - protein, 13 aa, synthetic construct
GGCLAIBGGVIAC

SEQ ID NO: 10 - protein, 13 aa, synthetic construct
GGCIAIBGLVAGC

SEQ ID NO: 11 - protein, 13 aa, synthetic construct
GGCVAIBGLIAGC

SEQ ID NO: 12 - protein, 13 aa, synthetic construct
GGCAAIBLVGIGC

SEQ ID NO: 13 - protein, 13 aa, synthetic construct
GGCLAIBGIVGAC

SEQ ID NO: 14 - protein, 13 aa, synthetic construct
GGCGAIBVILAGC

SEQ ID NO: 15 - protein, 13 aa, synthetic construct
GGCIAIBGVLAGC

SEQ ID NO: 16 - protein, 13 aa, synthetic construct
GGCVAIBLGAIGC

SEQ ID NO: 17 - protein, 13 aa, synthetic construct
GGCGAIBAVLGIC

SEQ ID NO: 18 - protein, 13 aa, synthetic construct
GGCVAIBGAILGC

SEQ ID NO: 19 - protein, 13 aa, synthetic construct
GGCGAIBLVGIAC

SEQ ID NO: 20 - protein, 13 aa, synthetic construct
GGCIAIBVAGLGC

SEQ ID NO: 21 - protein, 13 aa, synthetic construct
GGCLAIBGVGAIC

SEQ ID NO: 22 - protein, 13 aa, synthetic construct
GGCVAIBAGILGC

SEQ ID NO: 23 - protein, 13 aa, synthetic construct
GGCGAIBVLGAIC

SEQ ID NO: 24 - protein, 13 aa, synthetic construct
GGCLAIBGAIVGC

SEQ ID NO: 25 - protein, 13 aa, synthetic construct
GGCIAIBGVLGAC

SEQ ID NO: 26 - protein, 13 aa, synthetic construct
GGCVAIBGALIGC

SEQ ID NO: 27 - protein, 13 aa, synthetic construct
GGCGAIBAVILGC

SEQ ID NO: 28 - protein, 13 aa, synthetic construct
GGCIAIBLVGAGC

SEQ ID NO: 29 - protein, 13 aa, synthetic construct
GGCIAIBLVGAGC

SEQ ID NO: 30 - protein, 13 aa, synthetic construct
GGCVAIBGIGLAC

SEQ ID NO: 31 - protein, 13 aa, synthetic construct
GGCGAIBVAILGC

SEQ ID NO: 32 - protein, 13 aa, synthetic construct
GGCIAIBGVGALC

SEQ ID NO: 33 - protein, 13 aa, synthetic construct
GGCLAIBGIVGAC

SEQ ID NO: 34 - protein, 13 aa, synthetic construct
GGCVAIBALGIGC

SEQ ID NO: 35 - protein, 13 aa, synthetic construct
GGCGAIBLVIGAC

SEQ ID NO: 36 - protein, 13 aa, synthetic construct
GGCIAIBGVALGC

SEQ ID NO: 37 - protein, 13 aa, synthetic construct
GGCLAIBGAVIGC

SEQ ID NO: 38 - protein, 13 aa, synthetic construct
GGCLAIBGAVIGC

SEQ ID NO: 39 - protein, 13 aa, synthetic construct
GGCGAIBVLAIGC

SEQ ID NO: 40 - protein, 13 aa, synthetic construct
GGCIAIBGVLAGC

SEQ ID NO: 41 - protein, 13 aa, synthetic construct
GGCLAIBAVIGGC

SEQ ID NO: 42 - protein, 13 aa, synthetic construct
GGCVAIBGALIGC

SEQ ID NO: 43 - protein, 13 aa, synthetic construct
GGCGAIBLVAIGC

SEQ ID NO: 44 - protein, 13 aa, synthetic construct
GGCGAIBLVAIGC

SEQ ID NO: 45 - protein, 13 aa, synthetic construct
GGCIAIBGVLGAC

SEQ ID NO: 46 - protein, 13 aa, synthetic construct
GGCVAIBAIGLGC

SEQ ID NO: 47 - protein, 13 aa, synthetic construct
GGCVAIBAIGLGC

SEQ ID NO: 48 - protein, 13 aa, synthetic construct
GGCGAIBVLAGIC

SEQ ID NO: 49 - protein, 13 aa, synthetic construct
GGCIAIBGAVLGC

SEQ ID NO: 50 - protein, 13 aa, synthetic construct
GGCLAIBGIAVGC

SEQ ID NO: 51 - protein, 13 aa, synthetic construct
GGCVAIBLGAIGC

SEQ ID NO: 52 - protein, 13 aa, synthetic construct
GGCGAIBAVLGIC

SEQ ID NO: 53 - protein, 13 aa, synthetic construct
GGCIAIBGAVGLC

SEQ ID NO: 54 - protein, 13 aa, synthetic construct
GGCLAIBGVAGIC

SEQ ID NO: 55 - protein, 13 aa, synthetic construct
GGCVAIBGIALGC

SEQ ID NO: 56 - protein, 13 aa, synthetic construct
GGCGAIBVLGAIC

SEQ ID NO: 57 - protein, 13 aa, synthetic construct
GGCIAIBGALVGC

SEQ ID NO: 58 - protein, 13 aa, synthetic construct
GGCLAIBGVGAIC

SEQ ID NO: 59 - protein, 13 aa, synthetic construct
GGCVAIBAILGGC

SEQ ID NO: 60 - protein, 13 aa, synthetic construct
GGCGAIBLVGAIC

SEQ ID NO: 61 - protein, 13 aa, synthetic construct
GGCIAIBGVLGAC

SEQ ID NO: 62 - protein, 13 aa, synthetic construct
GGCLAIBAGVIGC

SEQ ID NO: 63 - protein, 13 aa, synthetic construct
GGCVAIBGILGAC

SEQ ID NO: 64 - protein, 13 aa, synthetic construct
GGCGAIBVLGIAC

SEQ ID NO: 65 - protein, 13 aa, synthetic construct
GGCIAIBGAVLGC

SEQ ID NO: 66 - protein, 13 aa, synthetic construct
GGCLAIBGIVGAC

SEQ ID NO: 67 - protein, 13 aa, synthetic construct
GGCVAIBAGILGC

SEQ ID NO: 68 - protein, 13 aa, synthetic construct
GGCGAIBVLAGIC

SEQ ID NO: 69 - protein, 13 aa, synthetic construct
GGCIAIBGVGALC

SEQ ID NO: 70 - protein, 13 aa, synthetic construct
GGCLAIBGVIGAC

SEQ ID NO: 71 - protein, 13 aa, synthetic construct
GGCVAIBGALGIC

SEQ ID NO: 72 - protein, 13 aa, synthetic construct
GGCGAIBVLAGIC

SEQ ID NO: 73 - protein, 13 aa, synthetic construct
GGCIAIBGVAGLC

SEQ ID NO: 74 - protein, 13 aa, synthetic construct
GGCLAIBGIVGAC

SEQ ID NO: 75 - protein, 13 aa, synthetic construct
GGCVAIBGAGLIC

SEQ ID NO: 76 - protein, 13 aa, synthetic construct
GGCGAIBVLGAIC

SEQ ID NO: 77 - protein, 13 aa, synthetic construct
GGCIAIBGVGLAC

SEQ ID NO: 78 - protein, 13 aa, synthetic construct
GGCLAIBGAVGIC

SEQ ID NO: 79 - protein, 13 aa, synthetic construct
GGCVAIBGLIGAC

SEQ ID NO: 80 - protein, 13 aa, synthetic construct
GGCGAIBVALGIC

SEQ ID NO: 81 - protein, 13 aa, synthetic construct
GGCIAIBGVAGLC

SEQ ID NO: 82 - protein, 13 aa, synthetic construct
GGCLAIBGIVGAC

SEQ ID NO: 83 - protein, 13 aa, synthetic construct
GGCVAIBGAIGLC

SEQ ID NO: 84 - protein, 13 aa, synthetic construct
GGCGAIBVLGAIC

SEQ ID NO: 85 - protein, 13 aa, synthetic construct
GGCIAIBGVLAGC

SEQ ID NO: 86 - protein, 13 aa, synthetic construct
GGCLAIBGVAIGC

SEQ ID NO: 87 - protein, 13 aa, synthetic construct
GGCVAIBAGLIGC

SEQ ID NO: 88 - protein, 13 aa, synthetic construct
GGCGAIBVLAGIC

SEQ ID NO: 89 - protein, 13 aa, synthetic construct
GGCIAIBGVGLAC

SEQ ID NO: 90 - protein, 13 aa, synthetic construct
GGCLAIBGAVGIC

SEQ ID NO: 91 - protein, 13 aa, synthetic construct
GGCVAIBGLAGIC

SEQ ID NO: 92 - protein, 13 aa, synthetic construct
GGCGAIBVALIGC

SEQ ID NO: 93 - protein, 13 aa, synthetic construct
GGCIAIBGVGALC

SEQ ID NO: 94 - protein, 13 aa, synthetic construct
GGCLAIBGAVGIC

SEQ ID NO: 95 - protein, 13 aa, synthetic construct
GGCVAIBGLIAGC

SEQ ID NO: 96 - protein, 13 aa, synthetic construct
GGCGAIBVLGAIC

SEQ ID NO: 97 - protein, 13 aa, synthetic construct
GGCIAIBGVAGLC

SEQ ID NO: 98 - protein, 13 aa, synthetic construct
GGCLAIBGVLAGC

SEQ ID NO: 99 - protein, 13 aa, synthetic construct
GGCVAIBGALGIC

SEQ ID NO: 100 - protein, 13 aa, synthetic construct
GGCGAIBVAILGC

SEQ ID NO: 101 - protein, 13 aa, synthetic construct
GGCIAIBGLVAGC

SEQ ID NO: 102 - protein, 13 aa, synthetic construct
GGCLAIBGVGLAC

SEQ ID NO: 103 - protein, 13 aa, synthetic construct
GGCVAIBGIGLAC

SEQ ID NO: 104 - protein, 13 aa, synthetic construct
GGCGAIBVLGIAC

SEQ ID NO: 105 - protein, 13 aa, unidentified
GGCIAIBGVLGAC

SEQ ID NO: 106 - protein, 13 aa, synthetic construct
GGCLAIBGAVIGC

SEQ ID NO: 107 - protein, 13 aa, synthetic construct
GGCVAIBGALIGC

SEQ ID NO: 108 - protein, 13 aa, synthetic construct
GGCAAIBGVLIGC

SEQ ID NO: 109 - protein, 10 aa, synthetic construct
GGCXVALAXC

SEQ ID NO: 110 - protein, 7 aa, synthetic construct
GGCNXMC

SEQ ID NO: 111 - protein, 12 aa, synthetic construct
GGCAVGLAGGXC